(19) United States
(12) Patent Application Publication
(10) Pub. No.: US 2024/0189239 A1
(43) Pub. Date: Jun. 13, 2024

(54) PROCESS FOR CONTINUOUS HOT MELT GRANULATION OF LOW SOLUBLE PHARMACEUTICALS

(71) Applicant: Merck Patent GmbH, Darmstadt (DE)

(72) Inventors: Thomas KIPPING, Darmstadt (DE); Finn BAUER, Darmstadt (DE); Nicole DI GALLO, Darmstadt (DE); Anja-Nadine KNUETTEL, Darmstadt (DE); Alessandro Giuseppe ELIA, Darmstadt (DE)

(73) Assignee: Merck Patent GmbH, Darmstadt (DE)

(21) Appl. No.: 18/284,883

(22) PCT Filed: Mar. 31, 2022
(86) PCT No.: PCT/EP2022/058528

§ 371 Date: Sep. 29, 2023

(30) Foreign Application Priority Data
Apr. 1, 2021 (EP) 21166645.8

(51) Int. Cl. A61K 9/20; A61K 9/14; A61K 9/16; A61K 45/06

(52) U.S. Cl. CPC A61K 9/2027; A61K 9/146; A61K 9/1635; A61K 9/1682; A61K 45/06

(57) ABSTRACT

The present invention relates to a process for loading a polymer with an active pharmaceutical ingredient in a melt granulation process and to the prepared product. More specifically the invention relates to a process of preparing granules which contain at least one active pharmaceutical ingredient and polyvinyl alcohol.

The present invention relates to a process for loading a polymer with an active pharmaceutical ingredient in a melt granulation process and to the prepared product. More specifically the invention relates to a process of preparing granules which contain at least one active pharmaceutical ingredient and polyvinyl alcohol.

### BACKGROUND

To achieve a more consistent dosage rate of the active pharmaceutical ingredient in pharmaceutical formulations, it is useful when the active pharmaceutical ingredient is present as a homogeneous dispersion or solution in carrier. Especially the solubility enhancement of low soluble drug substances is an important application for amorphous solid dispersions.

Solid dispersions are defined as being a dispersion of one or more active pharmaceutical ingredients in an inert solid matrix and can broadly classified as those containing a drug substance in the crystalline state or in the amorphous state [Chiou W. L., Riegelman S. Pharmaceutical applications of Solid dispersion systems; J. Pharm Sci. 1971, 60 (9), 1281-1301]. Solid dispersions containing pharmaceutical active ingredients in the crystalline state provide dissolution enhancement by simply decreasing surface tension, reducing agglomeration, and improving wettability of the active substance [Sinswat P., et al.; Stabilizer choice for rapid dissolving high potency itraconazole particles formed by evaporative precipitation into aqueous solution; Int. J. of Pharmaceutics, (2005) 302; 113-124]. While crystalline systems are more thermodynamically stable than their amorphous counterparts, the crystalline structure must be interrupted during the dissolution process, requiring energy. Solid dispersions containing an active pharmaceutical ingredient, this means a drug, dissolved at the molecular level, known as amorphous solid solutions, can result in a significant increase in dissolution rate and extent of supersaturation [DiNunzio J. C. et al. III Amorphous compositions using concentration enhancing polymers for improved bioavailability of itraconazole; Molecular Pharmaceutics (2008); 5(6):968-980]. While these systems have several advantages, physical instability can be problematic due to molecular mobility and the tendency of the drug to recrystallize. Polymeric carriers with high glass transition temperatures seem to be well suited to stabilize these systems by limiting molecular mobility.

As such, solid dispersions can be created by a number of methods, including, but not limited to, spray-drying, melt extrusion or thermokinetic compounding.

Hot melt extrusion (HME) recently gained acceptance in the pharmaceutical industry for the preparation of formulations comprising active pharmaceutical ingredients processed by extrusion. HME has been introduced as pharmaceutical manufacturing technology and has become a well-known process with benefits like continuous and effective processing, limited number of process steps, solvent free process etc. During hot melt extrusion mixtures of active pharmaceutical ingredients, thermoplastic excipients, and other functional processing aids, are heated and softened or melted inside of an extruder and extruded through nozzles into different forms.

Solid dispersions can also be created by granulation techniques. Granulation is a well-established pharmaceutical processing technique to agglomerate primary drug and excipient particles into larger secondary particles or granules. A great variety of both wet- and dry-granulation techniques are already established in pharmaceutical industry. In the field of continuous granulation, twin-screw granulation methods are the most promising technologies. Continuous twin-screw wet granulation (TSWG) is already frequently used to avoid flow and compression issues. Drawbacks here are stability and degradation issues by wet processing and proper control of the associated drying step.

Twin-screw melt granulation (TSMG) can provide an interesting alternative to wet granulation. Usually the agglomeration is initiated by a softened or molten binder instead of a granulation liquid, making this technology extremely suitable for moisture-sensitive drugs. Melt granulation is considered as a size enlargement process in which the addition of a binder that melts or softens at relatively low temperatures (usually at about 60° C.) is used to achieve agglomeration of solid particles. Different technologies are already well-established including spray-congealing and tumbling melt granulation.

From the technique of (hot) melt extrusion it is known that the polymers should have suitable properties, such as thermoplasticity, suitable glass transition temperature or melting point, thermostability at required processing temperature, no unexpected chemical interaction with active pharmaceutical ingredients etc.

### Object of the Present Invention

Common melt granulation techniques require the use of meltable binders. Those meltable binders are usually low melting substance that melt or soften at relatively low temperatures (50° C. to 90° C.), such as a low melting wax or a low melting polymer. The meltable binders are used to achieve agglomeration of solid particles during the granulation process. Typically, the processing temperature is set above the Tm or Tg of the polymeric binders but below the Tm of the drug substance. This maintains the drug in its crystalline state to minimize any physicochemical changes (Kittikunakorn N, Liu T, Zhang F. Twin-screw melt granulation: Current progress and challenges. International Journal of Pharmaceutics. 2020; 588:119670). Different types of hydrophilic and hydrophobic binders are already described. Usually the distribution of molten binders on the surface of the solid particles is driven by both mixing and compaction during melt granulation.

Also lipids are frequently used. However lower-melting binders bear the risk that the melting or softening of the binder also occurs during handling and storage of the agglomerates. Another problem is the limited number of suitable polymers.

It is therefore an object of the present invention to provide a melt granulation process to obtain an amorphous solid dispersion of an API in or on a polymer. It is a further object of the present invention to provide a composition that can be used in melt granulation processes without the need of a binder. It is a further object to provide a composition wherein the melt granulation process results in an amorphous solid solutions or dispersion of an API in or on a polymer without the need of a binder. It is a further object to provide granulation process parameters to archive the afore mentioned.

Furthermore, it is an object of the present invention to provide an extrusion method, wherein the product has beneficial properties, e.g. degree of amorphization or dissolution.

### Short Summary of the Invention

In common melt granulation processes a binder is used to form agglomerates of crystalline drug substances. Usually low melting polymers are preferred as the aim is in most cased only an agglomeration to increase the particle size.

Surprisingly it was found that a melt granulation process can be used to load a polymer with an active pharmaceutical ingredient (API) in its amorphous form. By utilizing polyvinyl alcohol (PVA) as a polymer during the melt granulation process the polymer can serve as a basis to stabilize an API in its amorphous form. Furthermore, it was surprisingly found that particular preferred results regarding stabilization of the amorphous form and dissolution of the API are obtained when the API is heated above its melting point. During the cooling step the liquid drug substance solidifies within the polymer or on the surface of the polymer and maintains its amorphous state to form an amorphous solid solution or dispersion.

Furthermore, it was found that the resulting particles have beneficial properties compared the particles prepared by HME.

It was further found that twin-screw melt granulation (TSMG) is particularly suitable as granulation process.

### DETAILED DESCRIPTION OF THE INVENTION

The present invention refers to a process of loading a polymer with an active pharmaceutical ingredient in a melt granulation process comprising the steps of:
a) kneading a mixture comprising at least one active pharmaceutical ingredient and polyvinyl alcohol in a heated screw barrel of an extruder, wherein the temperature in at least one zone along the length of the screw barrel is above the melting temperature of the at least one active pharmaceutical ingredient and below the decomposition temperature of polyvinyl alcohol to form a kneaded mixture, and
b) transporting the kneaded mixture through an outlet.

The “active pharmaceutical ingredient” or “API” may be found in the form of one or more pharmaceutically acceptable salts, esters, derivatives, analogues, prodrugs, and solvates thereof. As used herein, a “pharmaceutically acceptable salt” is understood to mean a compound formed by the interaction of an acid and a base, the hydrogen atoms of the acid being replaced by the positive ion of the base.

The term “polyvinyl alcohol” or “PVA” refers to a synthetic water-soluble polymer that has the idealized formula [CH₂CH(OH)]n. It possesses good film-forming, adhesive, and emulsifying properties. PVA is prepared from polyvinyl acetate, where the functional acetate groups are either partially or completely hydrolysed to alcohol functional groups. If not completely hydrolysed, PVA is a random copolymer consisting of vinyl alcohol repeat units —[CH₂CH(OH)]— and vinyl acetate repeat units —[CH₂CH(OOCCH₃)]—. The polarity of PVA is closely linked to its molecular structure. The hydrolysis degree and the molecular weight determine the molecular properties of PVA. As the degree of hydrolysis of acetate groups increases, the solubility of the polymer in aqueous media and also crystallinity and melting temperature of the polymer increase. However, at high hydrolysis degrees over 88%, the solubility of PVA decreases again. PVA is generally soluble in water, but almost insoluble in almost all organic solvents, excluding, in some cases, ethanol.

The typical PVA nomenclature indicates the viscosity of a 4% solution at 20° C. and the degree of hydrolysis of the polymer. For example, PVA 3-83 is a PVA grade with a viscosity of 3 mPas that is 83% hydrolysed, i.e. having 83% of vinyl alcohol repeat units and 17% of vinyl acetate repeat units. A skilled person is aware that a hydrolysis grade of 83% and a viscosity of 3 mPas encompasses calculated hydrolysis grades of 82.50% to 83.49% and calculated viscosities of 2.50 mPas to 3.49 mPas % according to common rounding methods. Viscosity according to the invention is measured as stated in USP 39 under Monograph “Polyvinyl Alcohol” with the method Viscosity-Rotational Method <912>. The degree of hydrolysis according to the invention is measured as stated in USP 39 under Monograph “Polyvinyl Alcohol” under “Degree of Hydrolysis”.

As the degree of hydrolysis increases, the solubility of the polymer in aqueous media increases, but also the crystallinity of the polymer increases. In addition to this, the glass transition temperature and melting temperature varies depending on its degree of hydrolysis, molecular weight and water content. For example, a PVA 4-88 with a loss of drying of ≤ 5.0% has a melting temperature of approximately 170 ºC, a glass transition temperature of approximately 40 to 45° C. and a decomposition temperature of >250° C. (Technical Information, Parteck® MXP). Thermal properties of a particular PVA or PVA grade can be measured using different methods. According to the present invention, glass transition temperature, melting temperature and decomposition temperatures are measured using Differential scanning calorimetry (DSC).

Polyvinyl alcohol is soluble in water, but almost insoluble in almost all organic solvents, excluding, in some cases, in ethanol. This aspect of the polymer makes it very difficult to form amorphous and solid dispersions through spray drying when the drug has also a limited solubility in aqueous media.

Polyvinyl alcohol according to the present invention can comprise any PVA grade.

In one embodiment the polyvinyl alcohol is composed of one or more grades of PVA of differing molecular weights and of differing grades of hydrolysis.

In one embodiment the polyvinyl alcohol has a hydrolysis degree of 72% to 90%, in particular 74% to 88%, in particular 80% to 90% and a viscosity of a 4% solution at 20° C. of 2 mPas to 40 mPas, in particular 3 mPas to 18 mPas.

In one embodiment the polyvinyl alcohol is selected from a list consisting of PVA 3-80, PVA 3-81, PVA 3-82, PVA 3-83, PVA 3-85, PVA 3-88, PVA 3-98, PVA 4-88, PVA 4-98, PVA 5-74, PVA 5-82, PVA 6-88, PVA 6-98, PVA 8-88, PVA 10-98, PVAPVA 13-88, PVA 15-99, PVA 18-88, PVA 20-98, PVA 23-88, PVA 26-80, PVA 26-88, PVA 28-99, PVA 30-98, PVA 30-92, PVA 32-88 and PVA 40-88.

In a further embodiment the polyvinyl alcohol is selected from a list consisting of PVA 3-80, PVA 3-81, PVA 3-82, PVA 3-83, PVA 3-88, PVA 4-88, PVA 5-74, PVA 5-88, PVA 8-88, and PVA 18-88.

In a further embodiment the polyvinyl alcohol is selected from a list consisting of PVA 3-80, PVA 3-81, PVA 3-82, PVA 3-83, PVA 4-88 and PVA 18-88.

In a further embodiment the polyvinyl alcohol is PVA 4-88. In a further embodiment the polyvinyl alcohol is PVA 3-82.

In a further embodiment the polyvinyl alcohol is cryo-milled. Preferably the PVA is cryo-milled to a particle size that is suitable for a melt granulation process. An exemplary commercially available PVA is Parteck® MXP.

The term “melting temperature” refers to the temperature of a substance at which it changes state from solid to liquid. At the melting temperature the solid and liquid phase exist in equilibrium. The melting temperature of a substance depends on pressure, according to the invention the melting point is specified at a pressure of 1 atmosphere. The melting temperature of a PVA grade depends on the hydrolysis degree and viscosity of the respective PVA grade. The melting temperature of PVAs in general is in the range of 180 to 220° C.

The term “glass transition temperature” refers to the gradual and reversible transition in amorphous materials, or in amorphous regions within semi-crystalline materials, from a hard and relatively brittle “glassy” state into a viscous or rubbery state as the temperature is increased. The glass transition temperature of a substance depends on pressure, according to the invention the glass transition temperature is specified at a pressure of 1 atmosphere. The glass transition temperature of a PVA grade depends on the hydrolysis degree and viscosity of the respective PVA grade. The glass transition temperature of PVAs in general is in the range of 40 to 80° C.

The term “decomposition temperature” refers to the temperature that causes a chemical decomposition wherein the heat is breaking chemical bonds. The decomposition temperature of a substance depends on pressure, according to the invention the decomposition temperature is specified at a pressure of 1 atmosphere. The decomposition temperature of a PVA grade depends on the hydrolysis degree and viscosity of the respective PVA grade. The decomposition temperature of PVAs in general starts at a temperature of 250° C.

In contrast to common hot melt granulation techniques where low melting binders are used for agglomeration of solid particles the process described here is performed at temperatures above the melting point of the active pharmaceutical ingredient and above the glass transition temperature of PVA. Experiments have shown that PVA is a very promising carrier because of its semi-crystalline nature. PVA can stabilize the amorphous phase of the active pharmaceutical ingredient in the solid dispersion.

In literature the use of lower-melting binders is preferred since higher-melting-point binders require high melting temperatures and can contribute to instability problems especially for heat-labile material such as heat-labile APIs (Melt granulation: An alternative to traditional granulation techniques; March 2013; Indian Drugs 50(3):5-13). According to the present invention a temperature is chosen that causes the API to melt during the granulation process. Surprisingly it was found that the molten API itself can be used as a binder and comes in close contact to the mobilized PVA. The close intermeshing within the granulation system assures a strong interaction between PVA and API and leads to a homogeneous distribution of the API in the polymer.

According to the invention, the minimum working temperature for obtaining an amorphous solid dispersion of the API in the melt granulation process is a temperature above the melting temperature of the API in at least in one zone along the length of the screw barrel. The maximum working temperature is the decomposition temperature of PVA. The glass transition temperature of PVA varies between 40° C. and 80° C. depending on the degree of polymerization and hydrolysis. Decomposition of most PVA grades start at approximately 250° C. Therefore, the method according to the invention can be used for APIs that have a melting point between 40° C. and 250° C. Typical working temperatures for obtaining an amorphous solid dispersion of an API in a PVA polymer are 140° C. to 230° C., preferably 170° ° C. to 210° C., more preferably 180° C. to 200° C.

The twin-screw melt granulation according to the present invention provides a major benefit compared to other processing technologies like hot melt extrusion (HME) where the plasticised melt is forced through a die which is attached at the end of the extruder barrel. In the process according to the invention the mixture is not passed through a die but through an outlet that is an opening which leads to PVA granules wherein HME leads to PVA pellets. The active pharmaceutical ingredient in the granules is better stabilized and/or shows an improved dissolution profile and can be easier processed to the final dosage form (tablets).

In one embodiment the temperature in at least one zone along the length of the screw barrel is above the melting temperature of the at least one active pharmaceutical ingredient and below the glass transition temperature and the decomposition temperature of polyvinyl alcohol.

If the temperature is above the melting temperature of PVA, the kneaded mixture that is transported through an outlet is in a molten state. Therefore, in step b) the kneaded mixture is transported through an outlet to obtain a melt/molten mixture.

In one embodiment the temperature in at least one zone along the length of the screw barrel is above the melting temperature of the at least one active pharmaceutical ingredient and below the melting temperature of polyvinyl alcohol, preferably at a temperature above the melting temperature of the at least one active pharmaceutical ingredient and between the glass transition temperature and the melting temperature of polyvinyl alcohol. In that embodiment the kneaded mixture that is transported through an outlet is in form of granules. Therefore, in step b) the kneaded mixture is transported through an outlet to obtain granules.

In a further embodiment the temperature in at least one zone along the length of the screw barrel is between 40° C. to 250° C., preferably 140° C. to 230° C., more preferably 170° C. to 210° C., most preferably 180º C to 200° C.

In a further embodiment the temperature as mentioned above is identical in all zones along the length of the screw barrel.

The term “melt granulation process” or “HMG” generally refers to a size enlargement process in which the addition of a binder that melts or softens is used to achieve agglomeration of solid particles in the formulation. The process utilizes materials that are effective as granulating agents when they are in the softened or molten state. In the pharmaceutical industry this process can be used for the preparation of fast release or sustained released dosage forms. A binder or meltable binder is usually a low melting substance that melt or soften at relatively low temperatures (50° ° C. to 90° C.), such as a low melting wax or a low melting polymer. The meltable binders are used to achieve agglomeration of solid particles during the granulation process. In contrast to HME, the mixture in the melt granulation process is transported through an outlet which is an opening but not a nozzle or die. This means that the outlet is dimensioned in such a way that it does not exert pressure on the kneaded mixture. This is in contrast to a die that leads to increase of velocity of a fluid at the expense of its pressure energy.

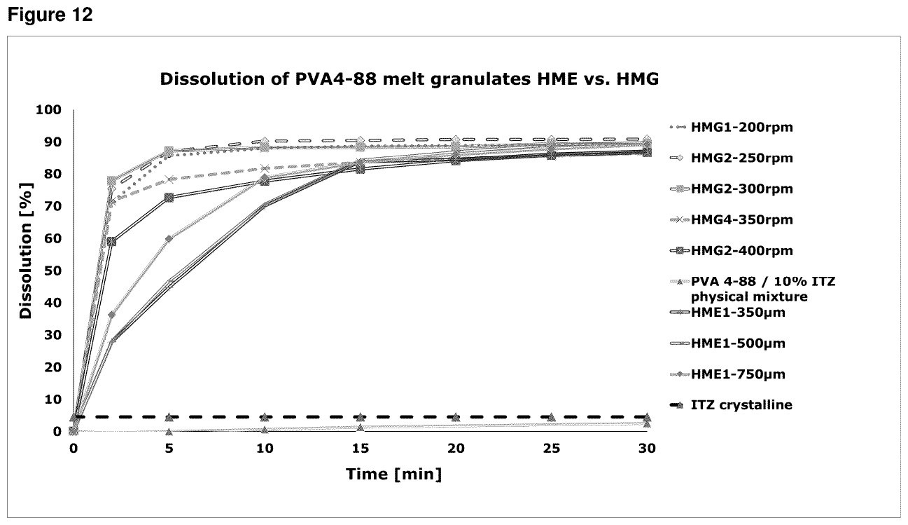

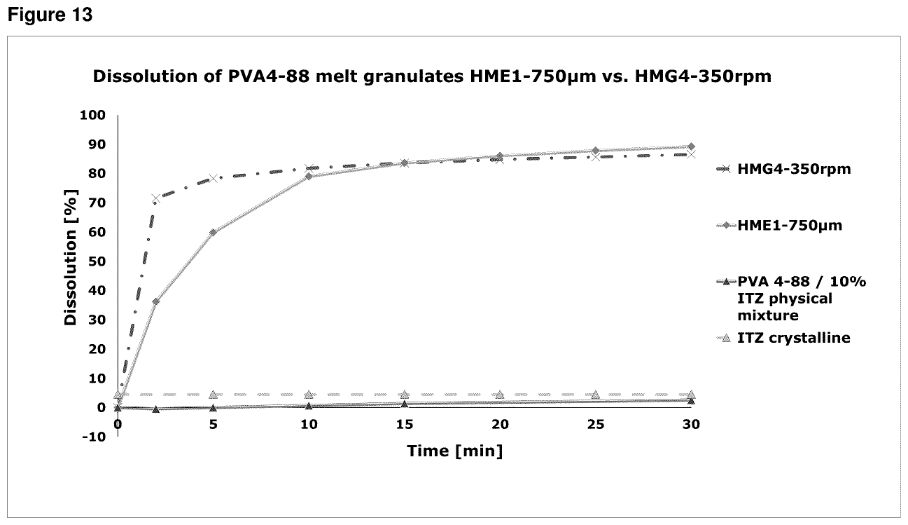

Products resulting from HMG differ from those prepared by HME. HME granules have an angular shape and a relatively even surface, wherein HMG granules have a more circular shape with a more uneven surface as can be seen by SEM measurement. HMG granules have a lower specific surface area. Unexpectedly, at a comparable particle size distribution (e.g. HME3-750 μm and HMG4-350 rpm) granules prepared by HMG show a faster API dissolution even though they have a lower specific surface area. Surprisingly, the dissolution of granules prepared by HMG is faster with increasing particle size as can be seen in FIG. 13. This is not the case with granules prepared by HME (FIG. 12).

According to the present invention no additional binder is needed. The degree of agglomeration depends on the temperature of the screw barrel. In the temperature range of the above described process (“above the melting point of the at least one active pharmaceutical ingredient and between the glass transition temperature and the decomposition temperature of polyvinyl alcohol”) the degree of agglomeration during the granulation process can be regulated by selection of a specific temperature. At a temperature below the melting temperature of PVA, a low degree of agglomeration can be detected. With increasing temperatures, the degree of agglomeration increases. Surprisingly it was found that additionally the molten and liquefied API can act as a binder to increase agglomeration.

Independent from the particle size and degree of agglomeration, it was surprisingly found that under the above described conditions the API is loaded on the PVA particles in an amorphous form and is stabilized in that form.

The amorphous solid dispersion can optionally contain further pharmaceutically acceptable components.

As used herein, the phrase “pharmaceutically acceptable” refers to all compounds, such as solvents, dispersion media, excipients, carriers, coatings, active agents, isotonic and absorption delaying agents, and the like that do not produce an allergic or similar untoward reaction when administered to humans in general. The use of such media and agents in pharmaceutical compositions is well known in the art.

In one embodiment the active pharmaceutical ingredient in the granules is dispersed in an amorphous form within the polyvinyl alcohol and/or on the surface of the polyvinyl alcohol.

The term “dispersed in an amorphous form” refers to a dispersion of an amorphous API within the polymer or on the surface of the polymer. Preferably, the amorphous API is distributed in a molecularly dispersed state on the polymer surface. Upon dissolution, formulations comprising an amorphous solid dispersion can reach higher solubilities in aqueous media than the crystalline API.

In one embodiment the API included in the pharmaceutical compositions of the present invention has a sufficient amount to be therapeutically effective. For a given API, therapeutically effective amounts are generally known or readily accessible by persons skilled in the art. Typically, the API may be present in the pharmaceutical composition in a weight ratio of API to PVA in the range of 1:99 to 90:10, preferably 5:95 to 60:40, most preferably 10:90 to 30:70.

The term “extruder” refers to a barrel containing one or multiple rotating screws that transport material down the barrel. Those extruders comprise (i) an opening though which material enters the barrel, which may have a hopper filled with the material(s) to be extruded or be continuously supplied in a controlled manner by one or more external feeder(s); (ii) a conveying (process) section, which comprises the barrel and the screw(s) that transport and, where applicable, mix the material and (iii) optionally downstream auxiliary equipment for cooling, cutting, classifying and/or collecting the finished product. According to the present invention, suitable extruders are single-screw extruders, twin-screw extruders or planetary roller extruders. Twin-screw extruders are preferred.

In one embodiment the melt granulation process is a twin-screw melt granulation process.

The term “twin-screw melt granulation” (TSMG) refers to a specific form of a melt granulation process. In a twin-screw melt granulation, a twin screw is used which consists of two intermeshing, co-rotating screws mounted on splined shafts in a closed barrel. Due to a wide range of screw and barrel designs, various screw profiles and process functions can be set up according to process requirements.

The twin screw is able to ensure transporting, compressing, mixing, cooking, shearing, heating, cooling, pumping, shaping with a high level of flexibility.

As used herein, the term “hot melt extrusion” or “HME” refers to process wherein active pharmaceutical ingredients, thermoplastic excipients and other functional processing aids are heated and softened or melted inside of an extruder and extruded through at least one nozzle or die into different forms.

The at least one active pharmaceutical ingredient (API) according to the invention is a biologically active agent that may be in the form of one or more pharmaceutically acceptable salts, esters, derivatives, analogues, prodrugs, and solvates thereof. The pharmaceutical composition may comprise one or more APIs.

As used herein, the terms “poorly soluble API”, “poorly water-soluble API” and “lipophilic API” refer to an API having a solubility such that the highest therapeutic dose of the particular API to be administered to an individual cannot be dissolved in 250 ml of aqueous media ranging in pH from 1 to 8 following the definition of low solubility according to the Biopharmaceutics Classification System (BCS) classes 2 and 4. APIs falling under BCS classes 2 or 4, respectively, are well known to persons skilled in the art. A typical example for a poorly soluble API of BCS class 2 is itraconazole (ITZ).

In one embodiment the active pharmaceutical ingredient is a poorly soluble API.

At the end of the process the product is preferably discharged in form of granules. These granules can easily be used for further processing steps, like milling, capsule filling, or direct compression with or without the addition of additional excipients. Particle sizes of the granules can be adapted by variation of process parameters.

As used herein, the term “granules” refers to a predominantly spherical, angular, near spherical or near angular structure of a macromolecular size, preferably having an average particle size between 20-2500 μm, more preferably between 50-2000 μm, most preferably between 100-1500 μm. The “average particle size” is defined as the equivalent diameter where 50% of the mass (of particles) of the sampled powder or granules have a smaller diameter. For the measurement of the particle size distribution, different methods are available. According to the present invention, the particle size distribution is measured by Dynamic Image Analysis (ISO 13322-2). In a particular embodiment, the particle size distribution is measured using a Camsizer X2 from Retsch GmbH, preferably with both camera systems CCD-B and CCD-Z being activated during the measurements, air pressure for dispersion being set to 50 kPa and slit width being set to 4 mm.

The concept of granulation process as carried out by melt granulation here is not only limited to twin screw melt granulation. Other thermal processing technologies, carried out batch-wise, are also possible, if the APIs as well as the polymer(s) can be processes under appropriate temperature conditions.

In the manner as described, itraconazole (ITZ) is processed as low soluble model active substance, and it is processed with polyvinyl alcohol using the disclosed melt granulation process to illustrate the invention. It is emphasized that the present invention is not limited to ITZ or low soluble APIs. The process can be performed with all APIs that have a melting temperature between 40° C. and 250° C. as stated above.

The present invention further refers to a method for producing an amorphous solid dispersion of at least one active pharmaceutical ingredient in PVA with the process as described above. The present invention further refers to a method for dispersing an amorphous active ingredient within PVA with the process as described above.

The present invention further refers to granules obtainable by a process described above. Granules obtained by the afore mentioned processes can be directly filled into sachets or capsules or further processed into tablets, capsules or multi-particulate systems.

Typically, the granules comprise at least the API and PVA. They can optionally contain further pharmaceutically acceptable components.

The at least one API and PVA may be present in the granules in a weight ratio of API to PVA in the range of 1:99 to 90:10, preferably 5:95 to 60:40, most preferably 10:90 to 30:70.

The granules preferably comprise at least 50% (w/w) of the API, more preferably at least 80%, most preferably at least 90% of the API in an amorphous form. Optionally the granules can be further milled to a defined particle size. Preferably the granules are milled to an average particle size between 50 μm to 300 μm.

The present invention further refers to tablets obtainable by a process described above.

While the making and using of various embodiments of the present invention are discussed in detail below, it should be appreciated that the present invention provides more applicable inventive concepts than described here in detail. The specific embodiments discussed herein are merely illustrative of specific ways to make and use the invention and do not delimit the scope of the invention.

Terms not defined herein have meanings as commonly understood by a person of ordinary skill in the areas relevant to the present invention. Terms such as “a”, “an” and “the” are not intended to refer to only a singular entity but include the general class of which a specific example may be used for illustration. The terminology herein is used to describe specific embodiments of the invention, but their usage does not delimit the invention, except as outlined in the claims.

### EXAMPLES

### I. Melt Granulation Process

### 1. Twin Screw Melt Granulation Process

700 g of a 10% mixture of crystalline Itraconazole and PVA 4-88 (Parteck® MXP) were fed into the barrel of a twin-screw extruder equipped with a granulation kit. Rotation speed and dosing rate were adapted until the targeted parameters were reached. After all heating zones reached their target temperature the granulation process was started.

Rotation speed of the screws was set to 300 rpm. Dosing rate was kept constant at 150 g/h. Temperature profiles of the individual heating zones are presented in Table 1.

TABLE 1
Temperature profiles showing the different heating zones of the extruder
during melt granulation at different target temperatures.

| | Temperature profiles | | | | | | | |
|---|---|---|---|---|---|---|---|---|
| Ex. No | HZ1 | HZ2 | HZ3 | HZ4 | HZ5 | HZ6 | HZ7 | HZ8 |
| 1.1 | 140 | 140 | 140 | 140 | 140 | 140 | 140 | 140 |
| 1.2 | 150 | 150 | 150 | 150 | 150 | 150 | 150 | 150 |
| 1.3 | 160 | 160 | 160 | 160 | 160 | 160 | 160 | 160 |
| 1.4 | 165 | 165 | 165 | 165 | 165 | 165 | 165 | 165 |
| 1.5 | 170 | 170 | 170 | 170 | 170 | 170 | 170 | 170 |
| 1.6 | 175 | 175 | 175 | 175 | 175 | 175 | 175 | 175 |
| 1.7 | 180 | 180 | 180 | 180 | 180 | 180 | 180 | 180 |
| 1.8 | 185 | 185 | 185 | 185 | 185 | 185 | 185 | 185 |
| 1.9 | 190 | 190 | 190 | 190 | 190 | 190 | 190 | 190 |
| 1.10 | 200 | 200 | 200 | 200 | 200 | 200 | 200 | 200 |

### 2. Powder Diffraction (PXRD)

Crystalline Itraconazole and granules obtained in Example 1 were measured with a powder diffractometer. Granules of Example 1 were milled in an IKA Tubemill 100 equipped with a 40 ml container for 20 sec at 25000 rpm. and sieved over a 250 μl sieve.

PXRD was measured with a Rigaku Miniflex 600 with the following settings:

X-Ray beam generation at 40 kV, 15 mA. A D/teX Ultra2 detector was used. The scan speed/duration time was set to 5 deg/min with a step width of 0.02 degree. The scan range was set to 3.0-50.0 degrees.

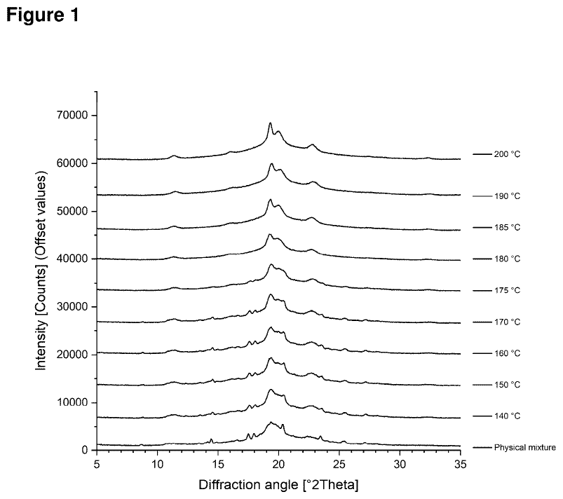

FIG. 1 is showing X-ray diffractograms of the different hot melt granules obtained at different processing temperatures as shown in Table 1.

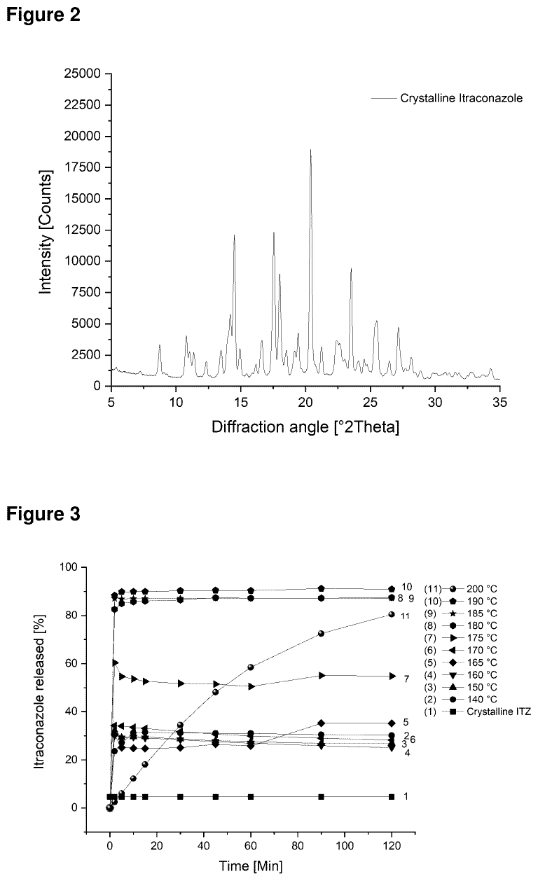

FIG. 2 is showing an X-ray diffractogram of crystalline itraconazole as used for the Twin screw melt granulation process of Example 1.

As can be seen in FIG. 1, the physical mixture of crystalline itraconazole and PVA still contains crystalline patterns, e.g. the peaks at a diffraction angle of approximately 14 and 21, that can clearly be linked to the crystalline itraconazole (see FIG. 2). The same patterns can also be observed at the samples until a processing temperature of about 170° C. At temperatures higher than 170° C. all the drug substance seems to be converted to its amorphous form. Itraconazole has a melting temperature of 166.2° C. Therefore, the experiment is showing that a temperature above the melting point is needed to obtain PVA particles that are loaded with amorphous Itraconazole.

### 3. Dissolution Measurement

For measuring the dissolution, granules as obtained in Example 1 were used directly without further processing. 3 samples per temperature setup were used. 500 mg 10% ITZ granulate were weighted (Mettler Toledo Delta Range XP105) which equals 50 mg ITZ API.

Dissolution was performed using a Sotax AT7 smart with an online Photometer Specord 200+ from Analytik Jena. As a medium 900 ml SGF.sp (20 g NaCl, 800 ml 0.1 M HCl ad 10.0 L VE-water) at 37° C.±0.5 was used. The following settings were used: Rotation speed 75 rpm; Paddle method; Pre-filter: Glass Mircofiber Filters GE Whatmann GF/D Diameter 25 mm, 5 mm HELMA flow-through cuvette, Sampling Points: 5,20,35,50,60,120 Min.

FIG. 3 shows the dissolution profiles of Itraconazole from granules obtained in Example 1 compared to the crystalline form of Itraconazole.

FIG. 3 demonstrates that crystalline itraconazole (line 1) shows a very low solubility in the release medium. The granulation with polyvinyl alcohol at a temperature between 140° C. to 170° C. increases the solubility of the compound (lines 2 to 6). At 175° C. the solubility suddenly increases. With higher temperature solubility is further enhanced. An immediate release can be observed for temperatures of 180° C. to 190° C. (lines 8 to 10). With a further temperature increase above the melting temperature of PCA (line 11; 200° C.) the drug release rates are delayed.

### 4. Particle Size Measurements

For measuring the particle size distribution, 1 to 3 g of the granules as obtained in Example 1 were used directly without further processing. Particle sizes were measured using a Camsizer X2 from Retsch GmbH. Both camera systems CCD-B and CCD-Z were activated during the measurements. Air pressure for dispersion was set to 50 kPa. Slit width was set to 4 mm. Cumulative distributions of the volume percent are given in Table 2.

TABLE 2
Cumulative size distributions (% passing based on volume).

| | Overview particle sizes [μm] | | | | | | | | | |
|---|---|---|---|---|---|---|---|---|---|---|
| | Exp | | | | | | | | | |
| | 1.1 | 1.2 | 1.3 | 1.4 | 1.5 | 1.6 | 1.7 | 1.8 | 1.9 | 1.10 |
| Q3 [%] | 140° C. | 150° C. | 160° C. | 165° C. | 170° C. | 175° C. | 180° C. | 185° C. | 190° C. | 200° C. |
| 5 | 257.8 | 26.8 | 20.2 | 21.1 | 21.5 | 26.9 | 29.7 | 33.1 | 57.4 | 695.3 |
| 10 | 349.9 | 40.3 | 27.7 | 28.4 | 28.5 | 34.3 | 37.4 | 41.6 | 77.7 | 878.7 |
| 20 | 462 | 69.3 | 40.8 | 42.5 | 39.5 | 49.8 | 51.7 | 61 | 115 | 1039.4 |
| 25 | 510.8 | 84.7 | 48.3 | 51.1 | 45.4 | 59.3 | 59.8 | 74 | 136.4 | 1157 |
| 30 | 558.4 | 101.8 | 56.2 | 61.6 | 53.4 | 70.4 | 69.9 | 90.8 | 163.2 | 1243 |
| 50 | 742.7 | 172.9 | 104.4 | 121.8 | 102 | 126.7 | 129.2 | 287 | 584.9 | 1433.5 |
| 75 | 1006 | 314.8 | 212.2 | 237.8 | 207.7 | 282.8 | 357.2 | 986.6 | 1199.2 | 1773 |
| 90 | 1228.3 | 501.3 | 367.6 | 385.2 | 363.9 | 535.9 | 713.3 | 1253.8 | 1472.6 | 2083.8 |
| 95 | 1328.7 | 628.7 | 476.5 | 487.3 | 486 | 685 | 857.9 | 1394.3 | 1567.5 | 2181.3 |
| 99 | 1649 | 859.2 | 664.7 | 720 | 789.9 | 943.4 | 1050.5 | 1706.5 | 1692.2 | 2234.5 |
| 100 | 1800.4 | 1169.8 | 1073.2 | 1167.1 | 1262.1 | 1194.9 | 1389.2 | 2034.2 | 1931 | 2352.8 |

II. Comparison Hot Melt Extrusion Vs. Hot Melt Granulation

### 1. Hot Melt Extrusion (HME)

540.15 g Parteck MXP and 60.0 g Itraconazole were weight in a mixing vessel and mixed for 5 minutes in a tubular mixer.

The Polymer/API mixture (ratio: 90/10) was then filled in the gravimetric twin screw feeder (Thermo Fisher scientific, Karlsruhe, Germany) and the max. dosing rate was measured. Max. dosing rate: 0.885 kg/h

The extrusion was conducted with a Pharma 11 twin screw extruder (Thermo Fisher scientific, Karlsruhe, Germany) with a screw speed of 250 rpm and a feed rate of 0.15 kg/h. Torque was 12% of max. possible torque (max. torque=12 Nm).

A round hole nozzle with 2.0 mm diameter was installed, also a vent port was installed.

The obtained white, opaque filament was transported via conveyor belt (Brabender GmbH & Co.KG., Duisburg, Germany). Conveyor belt speed was set to 1,49. Colling of the filament took place at room temperature. Filament was then cut by a Pelletizer (Brabender GmbH & Co.KG., Duisburg, Germany) and the white granules were collected.

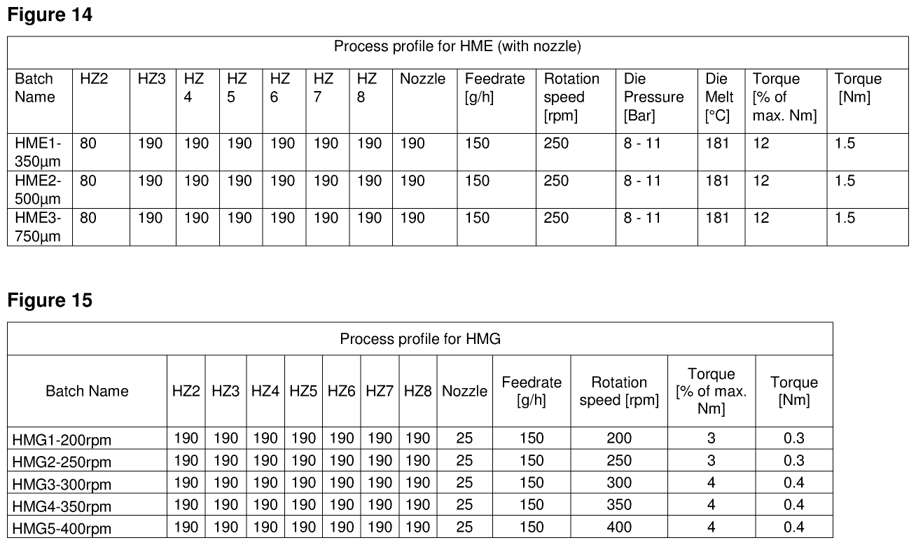

The process profiles for hot melt extrusion (with nozzle) are shown in FIG. 14.

### Milling:

The three batches of granules from the experiment with each 60 g were frozen with liquid nitrogen. The frozen granules were then milled with a ZM200 ultracentrifugally mill (RETSCH GmbH, Haan, Germany) at following conditions:
18000 rpm
12-teeth rotor
1^{st} batch with 350 μm distance sieve (batch HME1-350 μm)
2^{nd} batch with 500 μm distance sieve (batch HME1-500 μm)
3^{rd} batch with 750 μm distance sieve (batch HME1-750 μm)
cassette with cyclone

### 2. Hot Melt Granulation (HMG)

450.0 g Parteck MXP and 50.1 g Itraconazole were weight in a mixing vessel and mixed for 5 minutes in a tubular mixer.

The Polymer/API mixture (ratio: 90/10) was then filled in the gravimetric twin screw feeder Thermo Fisher scientific, Karlsruhe, Germany) and the max. dosing rate was measured. Max. dosing rate: 0.705 kg/h

The hot melt granulation was conducted with a Pharma 11 twin screw extruder (Thermo Fisher scientific, Karlsruhe, Germany), equipped with twin screw granulation kit.

Granulation was conducted at 190° ° C. at screw speeds of 200 rpm, 250 rpm, 300 rpm, 350 rpm and 400 rpm. Granules at given rpm were collected for 15 minutes. After each new setpoint granules were discarded for another 10 minutes before collecting new batch.

The process profiles for hot melt granulation are shown in FIG. 15.

### 3. SEM Measurement

### Sample Preparation:

A small amount of powder is prepared on an aluminum sample holder covered with electrically conductive double-sided adhesive tape. Non-adhered particles are removed by compressed air or bellows to prevent that loose particles contaminate the high vacuum chamber of the SEM. To avoid electrostatic charge up, the sample is coated with ˜10 nm platinum before measurement (If the particles are slightly wet a drying at 10-2 bar in the sputter system prior to sputtering is advised). The prepared sample is then transferred into the SEM and measured under high vacuum.

### SEM Instrumentation:

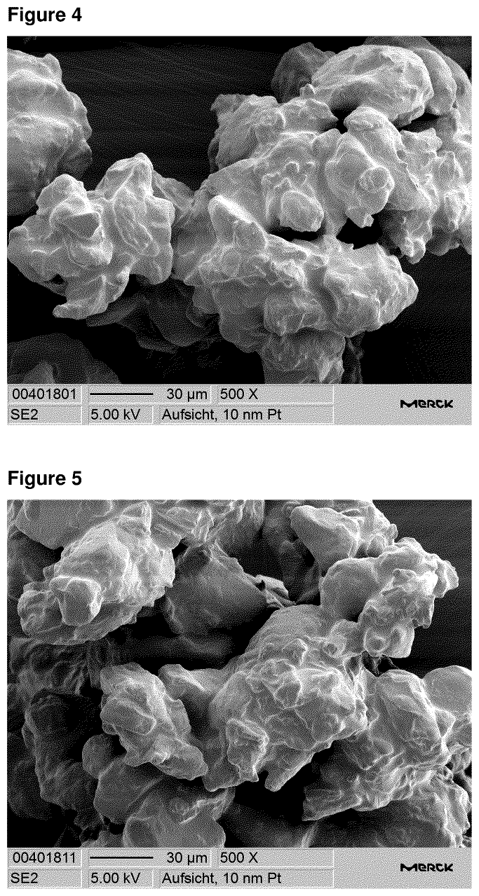

ZEISS Supra 35/LEO 1530, field emission cathode, resolution up to 2 nm, high vacuum, magnification 20× to 500.000×, voltage 0.1 kV to 30 kV, Inlens detector, Everhart-Thornley detector, 4-Quadrant BSE detector. FIG. 4 to FIG. 11 show SEM images of the particles from the batches as described above.

FIG. 4: SEM Image HMG1-200 rpm at 500× magnification

FIG. 5: SEM Image HMG2-250 rpm at 500× magnification

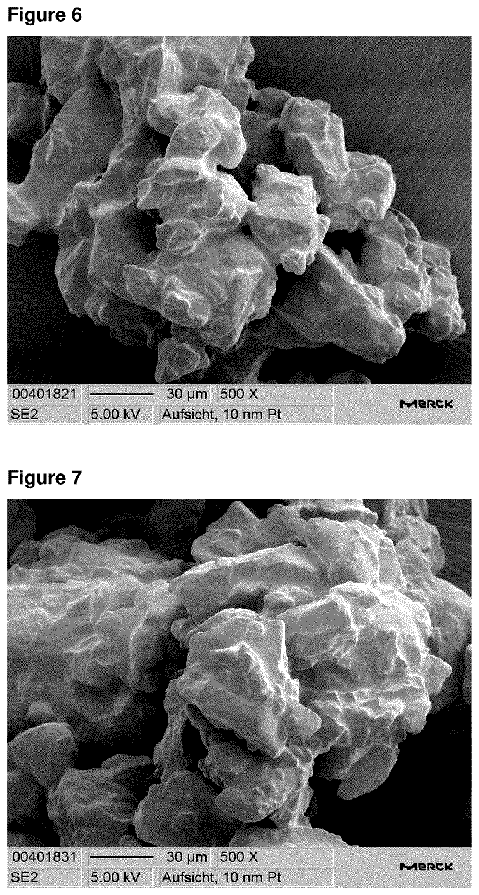

FIG. 6: SEM Image HMG3-300 rpm at 500× magnification

FIG. 7: SEM Image HMG4-350 rpm at 500× magnification

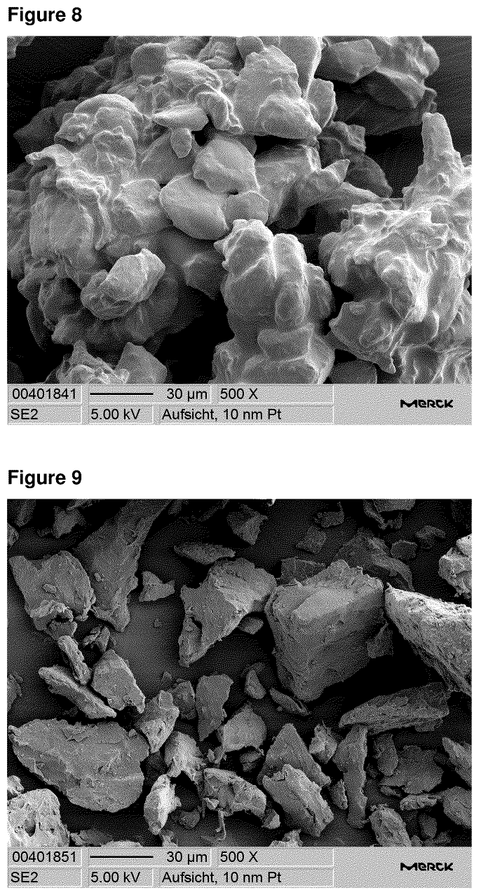

FIG. 8: SEM Image HMG5-400 rpm at 500× magnification

FIG. 9: SEM Image HME1-350 μm at 500× magnification

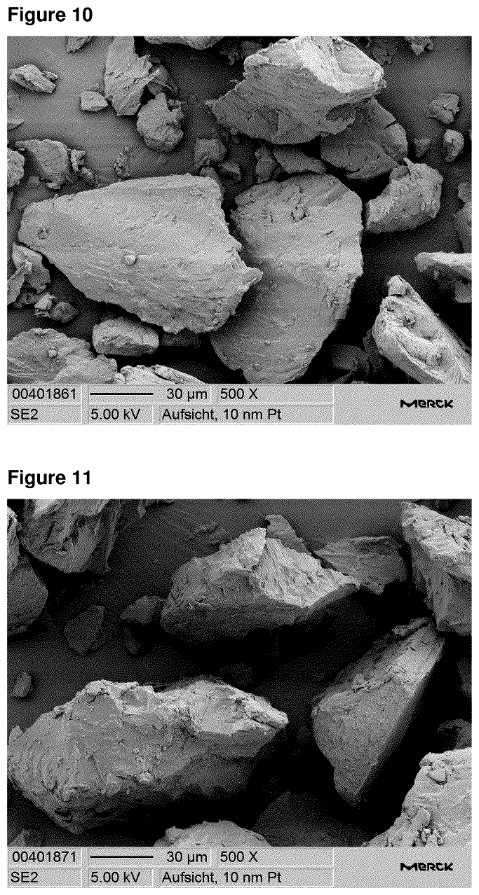

FIG. 10: SEM Image HME2-500 μm at 500× magnification

FIG. 11: SEM Image HME3-750 μm at 500× magnification

### 4. Dissolution Measurement:

Sample preparation: Use unmilled Samples: n=3 (per granule)

Weighing of 500 mg 10% ITZ granulate equals 50 mg ITZ API (Used balance: Mettler Toledo Delta Range XP105)

Dissolution was performed using a Sotax AT7 smart with an online Photometer Specord 200+ from Analytik Jena

Medium: 900 ml SGF.sp (20 g NaCl, 800 ml 0.1 M HCl ad 10,0 L VE-water) at 37° C.±0,5

Rotation speed 75 rpm; Paddle method; Pre-filter: Glass Mircofiber Filters GE Whatmann GF/D Diameter 25 mm 5 mm HELMA flow-through cuvette

### Sampling Points: 5, 20, 35, 50, 60, 120 Min

FIG. 12 shows the dissolution of the PVA4-88 particles of the different batches in 900 ml SGF, 75 rpm, Paddle with 50 mg API. FIG. 13 shows a comparison of the dissolution of batches HME3-750 μm and HMG4-350 rpm in 900 ml SGF, 75 rpm, Paddle with 50 mg API.

It can be seen that HMG4-350 rpm has a faster dissolution compared to HME3-750 μm. Both batches have a comparable particle size distribution (see example II 8.). In general HMG granules with higher particle sizes show a faster dissolution wherein this is the opposite for HME granules.

5. PXRD measurAement

Granules were milled in an IKA Tubemill 100 equipped with a 40 ml container for 20 sec at 25000 rpm. Sieved over a 250 μl sieve

SI-Low Backround sample holder

### PXRD Method:

Rigaku Miniflex 600
X-Ray 40 kV, 15 mA
Goniometer MiniFlex 600
Attachment ASC-8
Filter K-beta (×1.5)
Detector D/teX Ultra2
Scan mode CONTINUOUS
Scan speed/Duration time 5.0000 deg/min
Step width 0.0200 deg
Scan axis Theta/2-Theta
Scan range 3.0000-50.0000 deg
Incident slit 0.625 deg
Length limiting slit 10.0 mm
Receiving slit #1 13.0 mm (Open)
Receiving slit #2 13.0 mm (Open)

### 6. Description Camsizer:

The sample was placed into the groove (1-3 g)

Create or assign corresponding method for sample

### Start Measurement

### Camsizer Method:

Funnelposition [mm]: 5
Groove breadth [mm]: 60
Slit width [mm]: 4
Dispersion pressure [kPa]: 50
Velocity adjustment
Size definition: xc_min
Sieve size: Pharm.Eur
Parameters: Cumulative distribution, Spherecity, breadth/length, Symmetry
Cameras: CCD-Basic/CCD-Zoom
Frame rate: 100% (1:1)
Finish measurement after 5000 pictures

### 7. Specific Surface Area Measurement

The specific surface area was measured by gas adsorption—BET method. The measurement was executed corresponding to DIN ISO 9277:2014-01 and ISO 9277:2010(E).

The degassing and measurement of the samples were carried out by the “ASAP 2420” instrument from Micromeritics Instrument Cooperation, which uses the static-volumetric principle.

A sample quantity of 1.6 g to 4.5 g was used. The samples were dried and degassed under vacuum at 40° C. for 20 hours. Krypton (molecular cross-sectional area: 0.2100 nm²) was used as adsorptive. In order to achieve a good correlation, the specific surface area was calculated based on an adsorption isotherm in the pressure range p/p₀ from 0.05 to 0.20/0.23 using a multipoint determination (7 or 8 points). The correlation coefficient was greater than 0.9999 and the BET parameter C in a range from 13 to 18 for all measurements

For the inspection equipment monitoring two reference materials were used: alumina (specific surface area: 0.22 m²/g, batch 152624, article 004-16816-00) and silica-alumina (specific surface area: 199 m²/g, batch A-501-71, article 004/16821/00), both distributed by Micromeritics Instrument Cooperation.

TABLE 5
Results specific surface area

| | | weight of | specific surface |
|---|---|---|---|
| Bach Number | Gas | sample (g) | area (m²/g) |
| HME1-350 μm | Kr | 3.7887 | 0.2493 |
| HME2-500 μm | Kr | 4.5178 | 0.2116 |
| HME3-750 μm | Kr | 4.1370 | 0.1623 |
| HMG1-200 rpm | Kr | 1.6723 | 0.0812 |
| HMG2-250 rpm | Kr | 2.7580 | 0.0817 |
| HMG3-300 rpm | Kr | 2.6744 | 0.0917 |
| HMG4-350 rpm | Kr | 2.5037 | 0.0917 |
| HMG5-400 rpm | Kr | — | — |
| Alumina Standard | Kr | 2.1928 | 0.2192 |
| (Krypton) | | | |

### 8. Psd(Laser) Method:

The particle size distribution was measured by laser diffraction spectroscopy corresponding to ISO 13320:2020(E).

The laser diffraction spectrometer “Mastersizer 2000” with the dry dispersion unit “Scirocco 2000” from Malvern Panalytical Ltd. was used.

A sample quantity of approx. 1.5 g was analyzed. The sample was dispersed in air (refractive index of 1) using a feed rate of 75%, a size of gap of 6 mm, and an air pressure of 3 bar. The range of obscuration rate is set to 0.1% to 10%. A sieve (diameter 2 mm) with 10 balls was used.

The light diffraction pattern was evaluated with the Fraunhofer model with a general-purpose analysis model. Table 6 shows the particle size distribution of the described batches. HME3-750 μm and HMG4-350 rpm have a comparable particle size distribution.

TABLE 6
Results particle size distribution

| | Dv(0.1)/ | Dv(0.5)/ | Dv(0.9)/ | Dv(0.95)/ |
|---|---|---|---|---|
| Bach Number | μm | μm | μm | μm |
| HME1-350 μm | 36 | 124 | 266 | 313 |
| HME2-500 μm | 52 | 161 | 335 | 393 |
| HME3-750 μm | 56 | 212 | 500 | 595 |
| HMG1-200 rpm | 88 | 312 | 898 | 1055 |
| HMG2-250 rpm | 74 | 202 | 788 | 965 |
| HMG3-300 rpm | 58 | 145 | 653 | 840 |
| HMG4-350 rpm | 53 | 126 | 533 | 722 |
| HMG5-400 rpm | 51 | 126 | 580 | 785 |

1. A process of loading a polymer with an active pharmaceutical ingredient in a melt granulation process comprising the steps of:
a) kneading a mixture comprising at least one active pharmaceutical ingredient and polyvinyl alcohol in a heated screw barrel of an extruder wherein the temperature in at least one zone along the length of the screw barrel is above the melting temperature of the at least one active pharmaceutical ingredient and below the decomposition temperature of the polyvinyl alcohol to form a kneaded mixture and
b) transporting the kneaded mixture through an outlet.

2. The process according to claim 1, wherein the outlet does not exert pressure on the mixture.

3. The process according to claim 1, wherein the active pharmaceutical ingredient in the granules is dispersed in an amorphous form within the polyvinyl alcohol.

4. The process according to claim 1, wherein the temperature in at least one zone along the length of the screw barrel is above the melting temperature of the at least one active pharmaceutical ingredient and below the melting temperature of the polyvinyl alcohol and wherein the kneaded mixture is transported through an outlet to obtain granules.

5. The process according to claim 1, wherein the granulation process is a twin-screw melt granulation process.

6. The process according to claim 1, wherein the polyvinyl alcohol has a hydrolysis degree of 72% to 90% and a viscosity of a 4% solution at 20° ° C. of 2 mPas to 40 mPas.

7. The process according to claim 1, wherein the polyvinyl alcohol is selected from a list consisting of PVA 3-80, PVA 3-81, PVA 3-82, PVA 3-83, PVA 3-85, PVA 3-88, PVA 3-98, PVA 4-88, PVA 4-98, PVA 5-74, PVA 5-82, PVA 6-88, PVA 6-98, PVA 8-88, PVA 10-98, PVAPVA 13-88, PVA 15-99, PVA 18-88, PVA 20-98, PVA 23-88, PVA 26-80, PVA 26-88, PVA 28-99, PVA 30-98, PVA 30-92, PVA 32-88 and PVA 40-88.

8. The process according to claim 1, wherein the polyvinyl alcohol is PVA 4-88.

9. The process according to claim 1, wherein the polyvinyl alcohol is cryo-milled.

10. The process according to claim 1, wherein the active pharmaceutical ingredient is present in the mixture in a weight ratio of active pharmaceutical ingredient to polyvinyl alcohol in the range of 1:99 to 90:10.

11. The process according to claim 1, wherein the active pharmaceutical ingredient has a poor solubility in water.

12. The process according to claim 1, wherein the granules are further milled to an average particle size between 50 μm to 300 μm.

13. The process according to claim 1, wherein the granules are further processed into tablets.

14. Granules obtainable by a process according to claim 1.

15. Tablets obtainable by a process according to claim 13.

16. The process according to claim 1, wherein the temperature in said at least one zone along the length of the screw barrel is between 40° C. to 250° C.

17. The process according to claim 1, wherein the temperature in said at least one zone along the length of the screw barrel is between 140° C. to 230° C.

18. The process according to claim 1, wherein the active pharmaceutical ingredient is present in the mixture in a weight ratio of active pharmaceutical ingredient to polyvinyl alcohol in the range of 10:90 to 30:70.

19. The process according to claim 4, wherein the granules have an average particle size between 20-2500 μm.

20. The process according to claim 4, wherein the granules comprise at least 50% (w/w) of the active pharmaceutical ingredient.
